(19) United States
(12) Patent Application Publication
(10) Pub. No.: US 2006/0226030 A1
(43) Pub. Date: Oct. 12, 2006

(54) Capacitative biosensor element and method for detecting hybridization events

(76) Inventors: Hans-Christian Hanke, Munchen (DE); Christian Paulus, Weilheim (DE); Meinrad Schienle, Ottobrunn (DE); Roland Thewes, Grobenzell (DE)

Correspondence Address: HARNESS, DICKEY & PIERCE, P.L.C., P.O.BOX 8910, RESTON, VA, 20195, US

(21) Appl. No.: 10/562,040

(22) PCT Filed: May 11, 2004
(86) PCT No.: PCT/DE04/00978

§ 371 Date: Apr. 13, 2006

(30) Foreign Application Priority Data
Jun. 23, 2003 (DE) 103 28 136.3

(51) Int. Cl. G01F 1/64; G01N 33/487

(52) U.S. Cl. 205/792; 204/403.01

(57) ABSTRACT

A sensor element is for detecting DNA single strands which are possibly contained in an analyte. The sensor element includes a substrate and at least two electrodes in and/or on the substrate. In a surface area of the substrate, catcher molecules are immobilized and are adapted to hybridize to DNA single strands that are possibly contained in an analyte. The DNA single strands include a label that has dielectric properties that are different from those of the analyte. The electrodes are coupled to a detection device for detecting a change in the capacitative portion of the impedance between the electrodes due to a label that is present in an area surrounding the electrodes as a result of the hybridization event.

This application is the national phase under 35 U.S.C. § 371 of PCT International Application No. PCT/DE2004/000978 which has an International filing date of May 11, 2004, which designated the United States of America and which claims priority on German Patent Application number DE 103 28 136.3 filed Jun. 23, 2003, the entire contents of which are hereby incorporated herein by reference.

### FIELD

The invention generally relates to a sensor element, a sensor array and/or a method for detecting particles possibly contained in an analyte.

### BACKGROUND

The prior art discloses impedance sensors for biosensorics, see [1] to [8], the measurement principle of which is based on the alteration of the impedance of a probe in the presence of particles to be detected.

A description is given below, referring to FIG. 1, of a DNA sensor disclosed in the prior art.

In the case of the biosensor element 100 shown in FIG. 1, a first electrode 102 made of gold and a second electrode 103 made of gold are formed on a substrate 101. The first and second electrodes 102, 103 are realized as interdigital electrodes, that is to say as interdigitated electrode structures. FIG. 1 shows a plan view of the biosensor element 100 and a cross-sectional view taken along a section line I-I′.

The functionality of the biosensor element 100 is described in more detail below, referring to FIG. 2A, FIG. 2B, on the basis of a consideration of an enlarged partial region 104 of the biosensor element 100.

FIG. 2A, FIG. 2B show that catcher molecules 200 are in each case immobilized on the electrodes 102, 103. The catcher molecules 200 are DNA single strands. Gold is often used as material for the electrodes 102, 103 since, in this case, the binding of catcher molecules 200 to the gold electrodes 102, 103 can be realized well by means of a bond between thiol end groups (SH) of the catcher molecules 200 and the gold material of the electrodes 102, 103 on account of the chemically favorable gold-sulfur bond.

In order to detect particles 203 possibly contained in an analyte 201, such an analyte 201 is brought into operative contact with the biosensor element 100. In the case of the DNA sensor described in this example, the particles 203 to be detected that are possibly contained in the analyte 201 are likewise DNA single strands. The analyte 201 is often an electrolytic solution which is intended to be examined for the presence of particles 203 to be detected.

A hybridization between catcher molecules 200 and particles 203 to be detected is effected only when catcher molecules 200 and particles 203 to be detected match one another in accordance with the key-lock principle (see FIG. 2B). Hybridization denotes a binding of the DNA single strands to the catcher molecules 200. If catcher molecules 200 and particles 203 to be detected are not complementary to one another, that is to say if the base sequences of the DNA single strands 200, 203 do not match one another, then hybridization is not effected (see FIG. 2A). The specificity of the biosensor element 100 is thus derived from the specificity of the catcher molecules 200 for hybridizing with very specific particles 203 to be detected.

For detecting the particles 203, the impedance Z 202 between the electrodes 102, 103 is detected as an electrical parameter in the case of the biosensor element 100. In the case of hybridization that has been effected, the value of the impedance changes since catcher molecules 200 and particles 203 to be detected, as DNA single strands, in each case have relatively poor electrical conductivity and, after the hybridization, displace the volume of the electrolytic analyte 201, which has relatively good electrical conductivity, from the volume surrounding the electrodes 102, 103. An alteration of the value of the impedance can thus be interpreted as a sensor event.

FIG. 3 once again shows a part of the biosensor element 100 with its partial region 104. FIG. 3 furthermore shows profiles of electric field lines 301 between the interdigital electrodes 102, 103 if an electrical voltage for operating the biosensor element 100 is applied to them. FIG. 3 depicts surrounding regions 300 of the electrodes 103, 102 in which, after a hybridization event that has been effected, the electrical properties change to a particularly great extent on account of the presence of particles 203 to be detected that have relatively poor electrical conductivity. FIG. 3 furthermore reveals that the profiles of the electric field lines 300 in the case of an interdigital electrode arrangement in accordance with FIG. 1 have lines of symmetry 302 and recur periodically. It is therefore justified hereinafter to consider just two adjacent electrodes 102, 103.

FIG. 4A shows a first equivalent circuit diagram 400 for the partial region 104, in which diagram the components of the biosensor element 100 are modeled in the form of concentrated circuitry components. From a circuitry standpoint, the biosensor element 100 contains a second electrode-electrolyte capacitance 401 C_{M} between the second electrode 103 and the electrolytic analyte 201. Furthermore, a second electrode-electrolyte resistance 402 R_{M} (nonreactive resistance) is shown connected in parallel with the second electrode-electrolyte capacitance 401.

Connected in series with the parallel-connected components 401, 402 are the parallel-connected components electrolyte capacitance 403 C_{E} and electrolyte resistance 404 R_{E} (nonreactive resistance), by which the electrical properties of the electrolytic analyte 201 are modeled. The parallel circuit comprising the components 403, 404 is connected in series with a parallel circuit comprising a first electrode-electrolyte capacitance 405 C_{M} and a first electrode-electrolyte resistance 406 R_{M} (nonreactive resistance). A theoretical description of such a biosensor element 100 often assumes that preliminarily only the values of the components C_{M} and R_{M} change in the case of a hybridization (components 401, 402, 405, 406, see FIG. 4A).

However, since there is not only a change in the electrode impedances on account of the hybridization-dictated changes in the electrical properties in direct proximity to the electrodes 103, 102, but there is also a change in the properties of a volume of the electrodes 103, 102 near the interface (see surrounding regions 300 in FIG. 3), the second equivalent circuit diagram 410 from FIG. 4B may be used for even more precise description of the biosensor element 100. In the case of the second equivalent circuit diagram 410, the elements C_{E} and R_{E} identifying the electrolyte 201 are also represented as quantities that are variable on account of a hybridization.

In order to detect the value of the impedance metrologically in the case of the biosensor element 100, by way of example an AC voltage V is applied to one of the electrodes 103, 102 by means of an AC voltage source 500, as shown in FIG. 5A. A terminal of the AC voltage source 500 and a terminal of the components 401, 402 are connected to the electrical ground potential 504. Furthermore, an AC current signal I resulting from the AC voltage at the electrodes 103, 102 is evaluated by way of a current detection unit 501. As an alternative, a signal, that is to say an electrical voltage, may in each case also be applied to both electrodes 103, 102. In this case, these signals are then in antiphase with respect to one another.

FIG. 5B shows a scenario in which the capacitances 401, 405 are assumed to be identical and in which the nonreactive resistances 402, 406 are assumed to be identical. In this case, the capacitances 401, 405 are combined to form an effective electrode-electrolyte capacitance 502 and the components 402, 406 are combined to form an effective electrode-electrolyte resistance 503 (nonreactive resistance).

The components C_{E} and R_{E} are illustrated as non-variable electrical parameters in FIG. 5A, FIG. 5B. If their change on account of a hybridization is intended to be concomitantly detected, the illustrations shown in FIG. 5C and FIG. 5D, respectively, arise, with components 403, 404 that are variable on account of a hybridization.

A distance d (shown in FIG. 1) between the electrodes 102, 103 typically lies in the sub-micrometer range. A biosensor element 100 may (as shown in FIG. 1) be provided in essentially rectangular fashion. [2], [9] and [10] describe circular arrangements, which may be favorable for reasons of fluidics (for the spotting process when applying the catcher molecules to the electrodes 102, 103). The external dimensions 1 (see FIG. 1) or the diameter of a biosensor element typically lies in the range of between less than 100 micrometers and a few tens of millimeters.

It holds true for the exciting AC voltage V of the AC voltage source 500 that it should have a peak value which should not exceed a specific maximum value. The biochemical or electrochemical conditions which are necessary for the operation of a biosensor element 100 are no longer fulfilled when such a maximum value is exceeded. If the electrode potential exceeds a specific value, then specific substances at an electrode may be oxidized. If the electrical potential falls below another threshold value, substances are reduced at the electrode. An undesirable oxidation or reduction may have the effect, inter alia, that the chemical bonds which can be entered into in the course of immobilization and hybridization are broken. Furthermore, electrolysis may commence at the sensor electrodes 102, 103, the electrolysis products bringing the chemical milieu required for operation of the sensors out of equilibrium. The absolute values of the critical potentials result from the composition and the concentration ratios of the chemical surroundings of the electrodes (immobilization layer, analyte, etc.).

Typical values for the exciting voltage lie in the range of a few 10 mV to in the region around 100 mV. The magnitude of the resulting measurement signal (e.g. electric current) is approximately directly proportional to the applied voltage.

There is often an interest in carrying out not just one test with a sensor but many tests on a suitable sample, the analyte 201, temporally in parallel. Miniaturized bio-/chemosensor arrays that can be realized on a chip serve for parallel detection of different particles 203 to be detected in the analyte 201 to be examined. The multiplicity of electrical sensor elements is arranged in large numbers on a chip made of glass, plastic, silicon or some other substrate material. Such sensor array chips including corresponding evaluation systems are afforded diverse applications in medical diagnosis technology, in the pharmacological industry, e.g. for pharmacological screening (“high throughput screening”, HTS), in the chemical industry, in foodstuffs analysis, in ecological and foodstuffs technology and analysis, etc.

The described sensor elements known from the prior art often have the disadvantage of a low sensitivity in the area of molecular or DNA sensor technology. This will be explained with reference to the illustration in FIG. 6A, FIG. 6B. Although the lateral extent d_{strand} of double-stranded DNA after hybridization (cf. FIG. 6B) is greater than that of single-stranded DNA (cf. FIG. 6A), it is often small relative to the distance d_{footprint} between adjacent molecules.

Therefore, the electrical properties of the volume under consideration are essentially determined by the properties of the electrolyte 201 and are determined only slightly by the properties of the molecules 200, 203. The relatively low sensitivity of known sensor elements is furthermore often based on the DNA molecules—irrespective of the fact of whether or not a hybridization has taken place—being permeated by the ions that contribute to the conductivity of the surrounding electrolyte.

A description is given below, referring to FIG. 7A, FIG. 7B, of how this problem is intended to be reduced in accordance with [4]. [4] proposes choosing the width of the electrodes 102, 103 and the distances between the electrodes 102, 103 to be as small as possible (typically 200 nanometers or less up to the order of magnitude of the molecules 200, 203). A higher sensitivity is expected in this case since the density of the field lines 301 which run through the relevant volume 300 in which the hybridization takes place is substantially greater than in the case of larger electrode widths and distances. FIG. 7A shows a biosensor element with relatively large electrode distance and width; electrode distance and width are reduced in the case of the biosensor element shown in FIG. 7B.

However, the problem of excessively low volume occupancy is solved only inadequately by reducing the electrode width and the electrode distances. Furthermore, it must be taken into account that although apparatuses for processing very small structure widths are provided from modern microelectronics, these are very expensive and optimized for the standard metals (copper, aluminum, tungsten) in microelectronics. Electron beam lithography, which permits the production of even smaller structure widths than by means of the standard lithography methods that are customary nowadays, allows only a sequential processing of the required structures, and does not permit temporally parallel processing, and is thus likewise unsuitable for cost reasons.

[11] discloses providing particles to be detected in an analyte with small metal balls as a label. Such small metal balls are produced from materials such as gold or silver and used with diameters of a few nanometers. In the case of the method for detecting DNA single strands which is disclosed in [11], catcher molecules are immobilized on a surface region between two electrodes. Molecules of the substance to be detected are provided with the gold labels. The sample is then brought into operative contact with the sensor element.

After a hybridization event that has been effected, the small metal balls, having good electrical conductivity, are also arranged in the region between the electrodes. In accordance with [11], after a hybridization event that has been effected, a silver-containing solution has to be brought into operative contact with the double strands generated on account of the hybridization, whereby intermediate regions between adjacent small metal balls are bridged with silver material, with the result that an electrically conductive bridge is produced between the two electrodes. As a result, the value of the nonreactive resistance between the two electrodes is significantly changed, which is detected metrologically as a measure of the hybridization event.

However, the sensor disclosed in [11] has the disadvantage that the production of an electrically conductive bridge using small metal balls and the additional method step of bridging adjacent gold labels with silver material are costly and technically difficult.

[12] discloses a biochip arrangement having a substrate, having at least one sensor arranged on or in the substrate and having an electrically conductive permeation layer.

### SUMMARY

At least one embodiment of the invention is based, in particular, on the problem of providing a sensor element, a sensor array and/or a method for detecting particles possibly contained in an analyte in which it is possible, with a reduced outlay, to detect particles to be detected with high detection sensitivity.

The problem is improved upon or even solved by use of a sensor element, by use of a sensor array and/or by use of a method for detecting particles possibly contained in an analyte.

The sensor element according to at least one embodiment of the invention is for detecting particles possibly contained in an analyte contains a substrate, at least two electrodes in and/or on the substrate and catcher molecules which are immobilized on a surface region of the substrate. In at least one embodiment, the catcher molecules are set up in such a way that they hybridize with particles to be detected that are possibly contained in an analyte, which particles have a label having different electrical properties than the analyte. Furthermore, the sensor element contains a detection device coupled to the electrodes and serving for detecting an alteration of the capacitive component of the impedance between the electrodes on account of labels situated in a region surrounding the electrodes owing to a hybridization event.

The sensor array according to at least one embodiment of the invention contains a plurality of sensor elements having the features described above which are formed in and/or on the substrate.

In the case of the method according to at least one embodiment of the invention for detecting particles possibly contained in an analyte, a sensor element having the features described above is used. In accordance with the method, the analyte is brought into operative contact with the catcher molecules immobilized on the surface region of the substrate in such a way that the catcher molecules hybridize with particles to be detected that are possibly contained in the analyte, which particles have a label having different electrical properties than the analyte. Furthermore, the detection device coupled to the electrodes is used to detect an alteration of the capacitive component of the impedance between the electrodes on account of labels situated in a region surrounding the electrodes owing to a hybridization event.

Clearly, a basic idea of at least one embodiment of the invention can be seen in the fact that the sensor element according to at least one embodiment of the invention makes use of the detection sensitivity on account of the use of particles to be detected with a label having different electrical properties than the analyte, and that the detection of hybridization events is effected by way of a measuring method which is not resistive, e.g. capacitive. With the use of sufficiently large-volume labels on particles to be detected, in the case of a hybridization event, an electrolytic analyte is displaced from the surrounding region of the electrodes of the sensor element and replaced by a material having a significantly different electrical property. The imaginary component of the impedance between the electrodes, in particular the capacitance, changes significantly as a result. This alteration of the capacitive component of the impedance is detected metrologically.

In contrast to the method disclosed in [11] according to at least one embodiment of the invention, it is dispensable for a continuous conductive connection to be produced between two measuring electrodes on account of labels on particles to be detected. This is due to the fact that, in contrast to [11], at least one embodiment of the invention involves detecting an alteration of the capacitive component of the impedance rather than the nonreactive resistance between two electrodes. Consequently, the formation of an electrically conductive connection that completely bridges the electrodes is not a prerequisite for the successful detection of hybridization events according to at least one embodiment of the invention, since the capacitive component of the impedance rather than the nonreactive resistance is detected.

In contrast to [11], according to at least one embodiment of the invention it is furthermore not absolutely necessary for the electrodes to be directly exposed to the electrolytic analyte. By way of example, on account of the capacitive measuring method of the invention, the electrodes may be covered with a passivation layer, so that the electrodes are protected from being adversely influenced by an electrolyte that is possibly chemically aggressive. The service life of the sensor element according to at least one embodiment of the invention is thereby increased.

Furthermore, it is not necessary in this case to use a specific material for the electrodes such as e.g. gold; it is possible to use all electrically conductive materials, which, by way of example, can be introduced into the production process more cost-effectively and more favorably in terms of production technology, or are already available in the production process. In contrast to at least one embodiment of the invention, in accordance with [11] the electrode always has to be in electrical operative contact with the electrolyte since a nonreactive resistance between the electrodes is detected. In accordance with [11], it is furthermore necessary, after a hybridization event that has been effective, for a silver-containing solution to be brought into operative contact with the double strands generated on account of the hybridization, as a result of which intermediate regions between adjacent small metal balls are bridged with silver material, so that an electrically conductive bridge is produced between the two electrodes. This complex method step is dispensable in the case of the solution according to at least one embodiment of the invention.

It should be noted that the labels having different electrical properties than the analyte may for example be metallically conductive or have poor electrical conductivity, or may have a particularly high relative permittivity. It is merely necessary for the capacitive component of the impedance between the electrodes to be subjected to a significant change in the presence of the labels in a surrounding region of the electrodes.

A distinction feature of the sensor element according to at least one embodiment of the invention with the use of metallically conductive labels compared with known sensor elements is that, in the case of a successful hybridization, the complex impedance between the electrodes decreases, or, if only the capacitive component is taken into consideration, the value of the capacitance increases, and the impedance thereof does not increase or the value of the capacitive components does not decrease.

On account of the introduction of the labels having the different electrical properties than the analyte, in the case of a metallic label made of a material having good electrical conductivity, the profile of the field lines is greatly influenced particularly in a surrounding region of the electrodes. In other words, the measurement effect is very large. Instead of labels or beads having very good electrical conductivity as label molecules, it is also possible to use such beads which, although they have a diameter similar to that of the readily conductive beads described previously, have a different electrical property. If the electrical resistance of such beads is substantially greater than the electrical resistance of the electrolyte and the relative permittivity is significantly smaller, successful hybridization gives rise to a decrease in the capacitive component of the impedance. Clearly, such a change in impedance is not associated with the concentration of the field lines between the beads, as in the case of metallically conductive labels, but rather with a displacement of the field lines from the volume occupied by the beads having poor electrical conductivity and having a low relative permittivity.

It is also possible to use beads or molecules which have poor electrical conductivity and have a very large relative permittivity. In this case, the impedance increases at low frequencies of an exciting signal, and the impedance decreases at high frequencies.

One advantage when using beads having poor electrical conductivity consists in the fact that an increase in the impedance can be limited to specific frequency ranges of an exciting signal, since the dielectric properties of the beads under consideration also play a part. The ratio of the desirable capacitive contributions relative to the resistive contributions can be set optimally by way of setting a suitable frequency.

Another advantage of the sensor element according to at least one embodiment of the invention is that a particularly small structure width of the electrodes is not required since the effect exploited is very highly pronounced particularly when using metallically conductive labels. It is therefore possible to produce the sensor element according to at least one embodiment of the invention using standard processes and without expensive special processes such as electron beam lithography.

The coupling chemistry used for the immobilization of catcher molecules is according to at least one embodiment of the invention preferably geared to guaranteeing a best or densest possible immobilization of the catcher molecules not only on but in particular also between the electrodes. The quality of the immobilization of the electrodes is rather of secondary importance. If the sensor according to at least one embodiment of the invention is produced on the basis of a silicon substrate (e.g. wafer, chip), it is possible to form the chip surface between adjacent sensors or between adjacent electrodes e.g. from the materials silicon oxide and/or silicon nitride. These materials are sufficiently well suited to the coupling of catcher molecules; furthermore, these materials can easily be modified and optimized in terms of their chemical constitution. By way of example, gold or platinum is a good choice for the electrode materials. Chemically inert materials (e.g. noble metals) are particularly advantageous. The sensor element of the invention can be produced by way of a robust and cost-effective production process.

Furthermore, it is possible to provide the electrodes in buried fashion or in a manner covered by means of a dielectric covering layer. As a result, the same surface is obtained between the electrodes and above the electrodes. Consequently, the coupling chemistry used for the immobilization of the catcher molecules only has to be adapted to one material. In particular, the entire biochemical system comprises one component fewer, is less complicated in this respect, and permits a simpler and more robust design.

In this case, therefore, the use of active CMOS chips is possible according to at least one embodiment of the invention without a high outlay since it is not necessary to integrate into a process a metal which is foreign to CMOS and fulfills the given biological requirements (e.g. gold).

Realizing the electrodes as buried electrodes furthermore results in a complete electrical isolation of electrolyte potential and electrode potentials. This is advantageous if an overall system is realized from electrolyte, potential-providing circuitry components for the electrolyte, sensors and circuits evaluating sensor signals. Each individual one of these components may optionally be provided on-chip or off-chip.

The sensor element may have an electrically insulating layer between the electrodes and the catcher molecules and/or on regions of the substrate between the electrodes. In this case, the electrodes are electrically isolated from the electrolyte, undesirable electrochemical conversions at the electrodes are avoided, and the electrodes are protected from an electrolyte that is possibly chemically aggressive.

The catcher molecules may be immobilized on or above the electrodes, on the one hand, and between the electrodes, on the other hand. In the case of an immobilization of the interspace between the electrodes on the substrate, it is possible to achieve a large alteration of the capacitive component of the impedance and a high detection sensitivity.

The sensor element may be set up as a biosensor element, in particular for detecting DNA molecules, proteins, oligonucleotides, etc.

The sensor element according to at least one embodiment of the invention is preferably set up as a monolithically integrated sensor element. In this case, electrical components for driving or reading the sensor element may be integrated in the substrate (e.g. silicon wafer or silicon chip). Consequently, the sensor element according to the invention can be realized with the advantages of modern silicon microelectronics, which enables an increased integration density and a particularly high detection sensitivity (for example on account of the digitization and/or preamplification of the measurement signal on-chip).

The sensor element may have two electrodes, and the detection device may be set up for detecting an electrical AC current signal on account of an AC voltage signal applied between the two electrodes. The two electrodes may be set up for example as interdigital electrodes (see FIG. 1) or as planar electrodes arranged one beside another or one in another. An electrical AC voltage signal can be applied by way of the detection device, and a sensor current altered owing to a hybridization event on account of the presence of the labels can be detected in order to determine the capacitive component of the impedance.

The sensor element may have two pairs of electrodes, and the detection device may be set up for detecting a current signal at one of the pairs and for detecting a voltage signal at the other of the pairs. Consequently, the sensor element may be realized as a four-pole sensor with two force electrodes and two sense electrodes (cf. FIG. 11 to FIG. 12B).

The catcher molecules can be arranged at such a distance from one another and/or the labels may have such a dimensioning that, in the case of hybridization events, the region between the electrodes is free of a continuous bridging by the labels. In contrast to the method disclosed in [11], therefore, according to at least one embodiment of the invention it is not necessary to realize a continuous electrically conductive connection between the electrodes by means of the labels. Even by way of a partial displacement of the electrolyte from the region between the electrodes on account of the labels of the particles to be detected, it is possible to achieve a sufficiently great change in the capacitive component of the impedance in order to obtain a signal that can be evaluated metrologically.

The labels may be formed from an electrically insulating material. In particular, the labels may have a relative permittivity which is greater than a relative permittivity of the analyte.

As an alternative, the labels may be formed from an electrically conductive material. In particular, the labels may be formed from small metallic balls having dimensions in the nanometers range.

Furthermore, it is possible to provide one portion of the labels from an electrically conductive material and another portion of the labels from a dielectric material.

Refinements of the sensor element also apply to the sensor array and to the method for detecting particles possibly contained in an analyte.

Example embodiments of the invention are illustrated in the figures and are explained in more detail below.

### BRIEF DESCRIPTION OF THE DRAWINGS

In the figures:

FIG. 1 shows a plan view and a cross-sectional view, taken along the section line I-I′ shown in FIG. 1, of a biosensor element in accordance with the prior art,

FIGS. 2A, 2B show cross-sectional views of a partial region of the biosensor element shown in FIG. 1 in two different operating states,

FIG. 3 shows a partial region of the biosensor element from FIG. 1 with a symmetrical field line profile,

FIGS. 4A, 4B show first and second equivalent circuit diagrams of a partial region of the biosensor element from FIG. 1,

FIGS. 5A to 5D show other equivalent circuit diagrams of a partial region of the biosensor element from FIG. 1,

FIGS. 6A, 6B show enlarged illustration of a partial region of the biosensor element from FIG. 1,

FIGS. 7A, 7B show schematic views of biosensor elements in accordance with the prior art with different structure dimensions,

FIGS. 8A, 8B show a biosensor element in accordance with a first example embodiment of the invention in two different operating states,

FIGS. 9A, 9B show schematic views of the electric field profile of the biosensor element in accordance with the first example embodiment of the invention in the two operating states as shown in FIGS. 8A, 8B,

FIGS. 10A, 10B show a biosensor element in accordance with a second example embodiment of the invention in two different operating states,

FIG. 11 shows a view of a biosensor element in accordance with a third example embodiment of the invention,

FIGS. 12A, 12B show different views of a biosensor element in accordance with a fourth example embodiment of the invention.

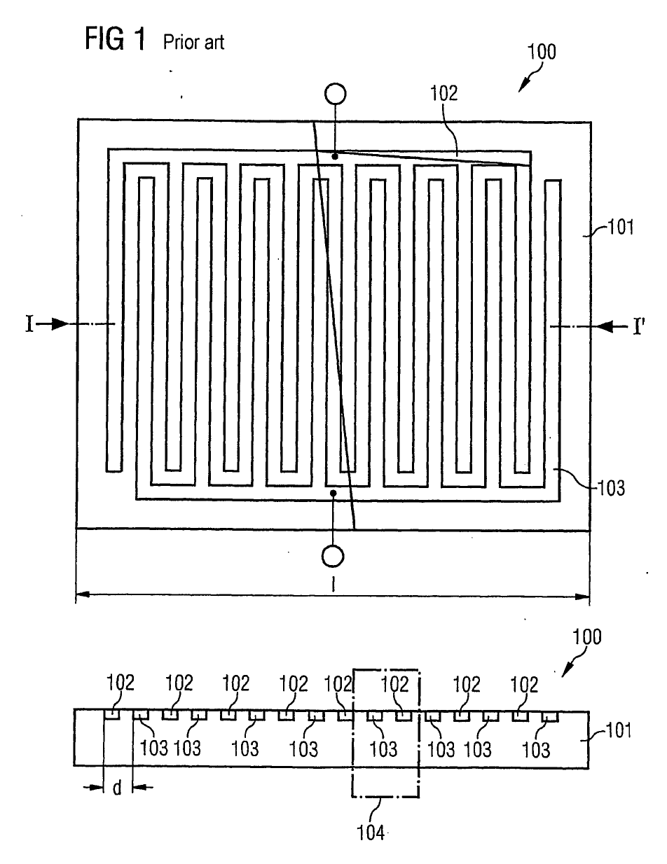

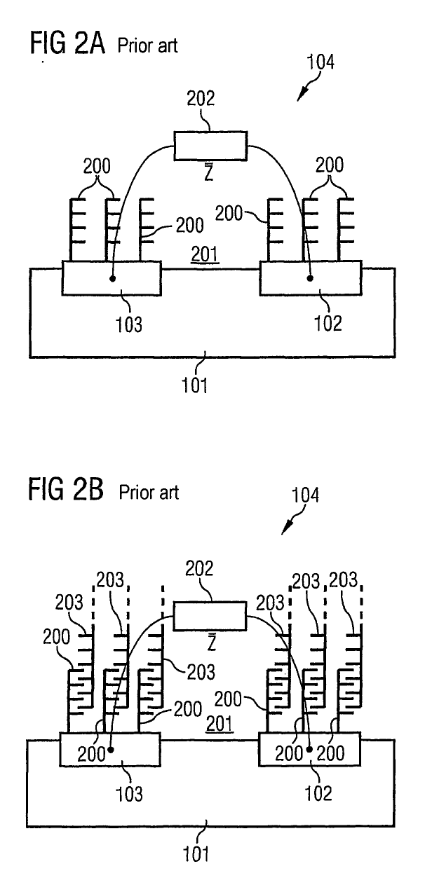

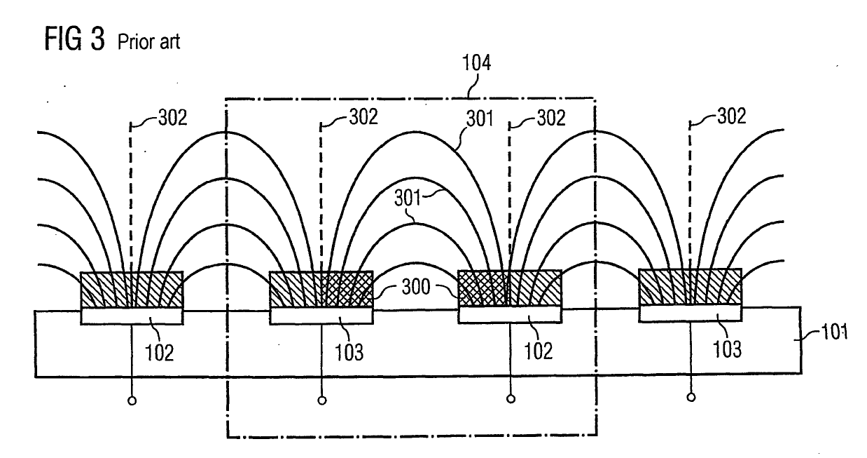

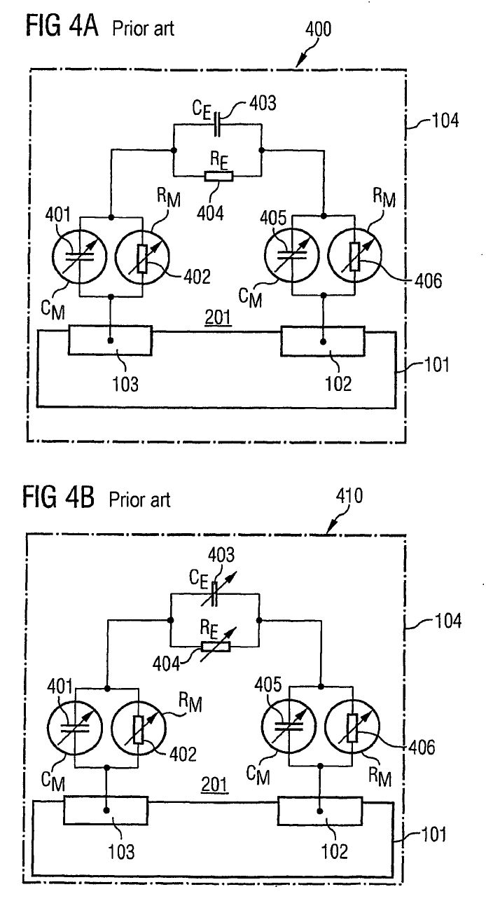

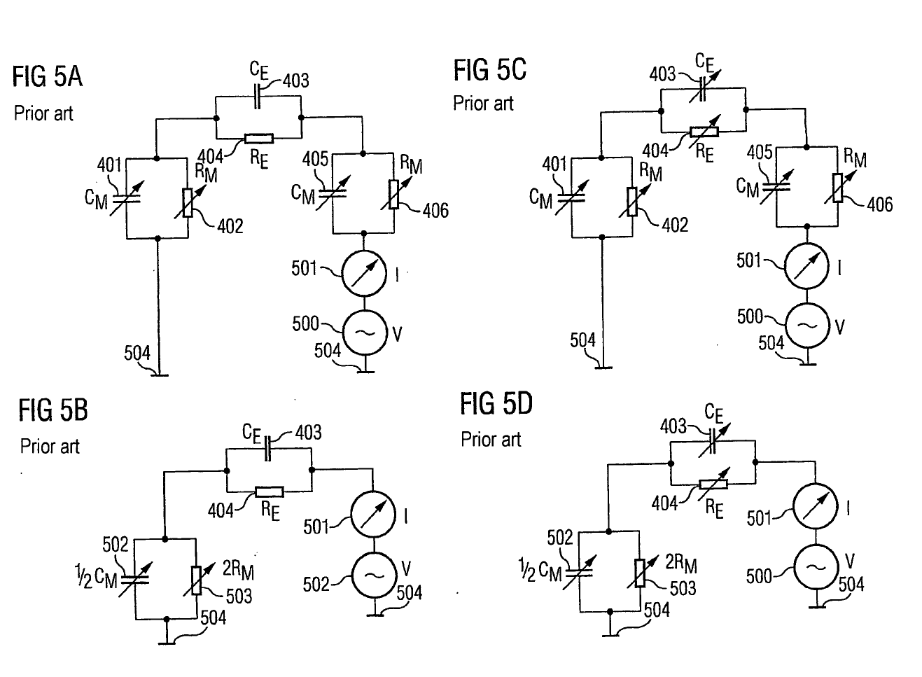

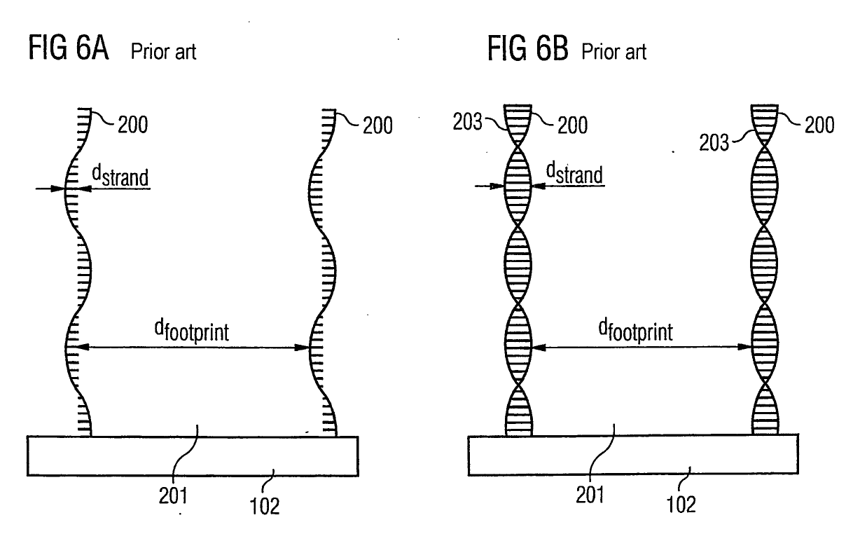

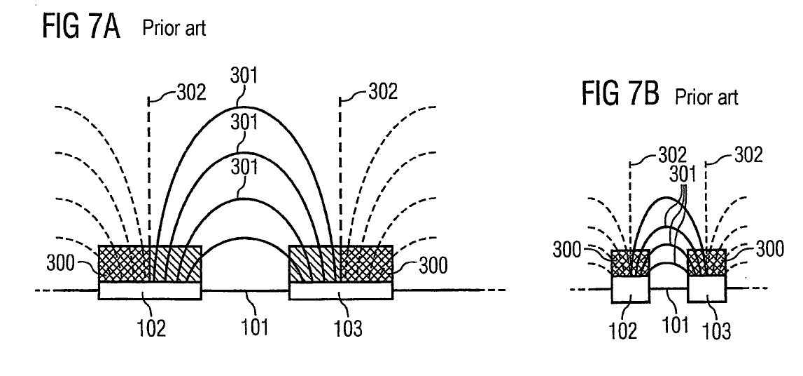

Identical or similar components in different figures are provided with identical reference numerals.

The illustrations in the figures are schematic and not to scale.

### DETAILED DESCRIPTION OF THE EXAMPLE EMBODIMENTS

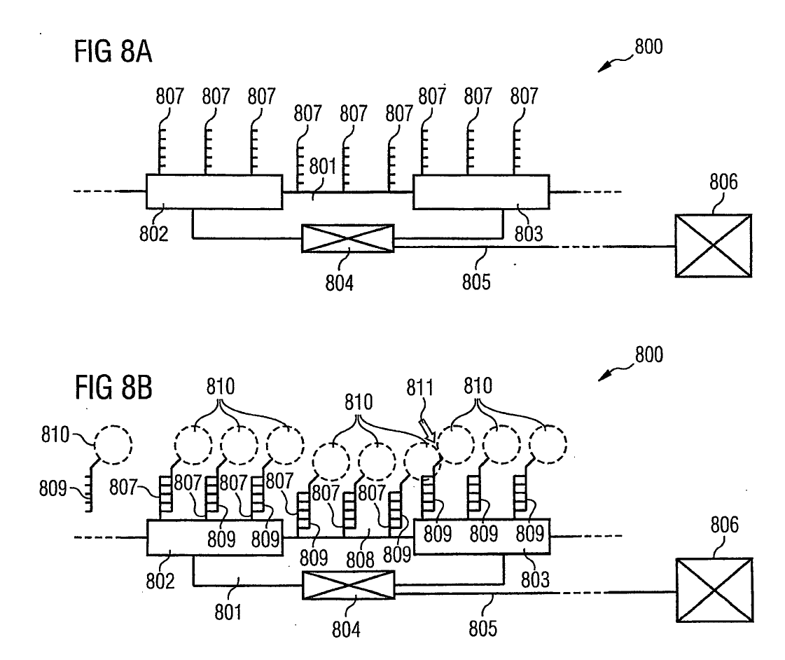

A description is given below, referring to FIG. 8A, FIG. 8B, of a biosensor element 800 in accordance with a first example embodiment of the invention.

The biosensor element 800 for detecting DNA single strands possibly contained in an analyte has a silicon substrate 801. A first gold electrode 802 and a second gold electrode 803 are formed on and in the silicon substrate 801. A detection device 804 is monolithically integrated in the silicon substrate 801. By way of the detection device 804, an AC voltage can be applied between the electrodes 802, 803 and a resulting AC current signal can be detected. The value of the capacitive component of the impedance or the alteration of such a value on account of a hybridization event can be detected from the detected AC current signal by means of the detection device. Such a sensor signal is preprocessed and amplified by the detection device 804 “on-chip” in the silicon substrate 801, that is to say near to the location of the sensor event, and communicated by way of a buried communication line 805 to an external evaluation unit 806 (realized off-chip) with respect to the silicon substrate 801.

DNA single strands are immobilized as catcher molecules 807 both on the gold electrodes 802, 803 and on the region of the silicon substrate 801 between the gold electrodes 802, 803.

FIG. 8A shows the biosensor element 800 in a first operating state before the biosensor element 800 is brought into contact with an analyte possibly containing particles to be detected.

FIG. 8B shows the biosensor element 800 after it has been brought into contact with an electrolytic analyte 808. The analyte 808 contains DNA single strands that are complementary to the catcher molecules 807, as particles 809 to be detected. Gold labels 810 having good electrical conductivity are bound to the particles 809 to be detected, as labels having significantly different electrical properties in comparison with the electrolyte.

In the case of the scenario shown in FIG. 8B, the base sequences of the catcher molecules 807 and of the particles 809 to be detected are complementary to one another, so that hybridization events occur (“match”). If the base sequences of catcher molecules 807 and particles 809 to be detected are not complementary to one another, no hybridization is effected (“mismatch”, not shown). After hybridization has been effected, as shown in FIG. 8B, the surrounding regions of the electrodes 802, 803 are partly taken up by the gold labels 810.

It should be noted that, in FIG. 8A, FIG. 8B, the distance between adjacent catcher molecules 807 is typically of the order of magnitude of 10 nanometers and the extent of the gold labels 810 is typically in the range of 2 to 7 nanometers. On account of the fact that the gold labels 810 having different electrical properties than the analyte 808 are present near the electrodes in a manner dictated by the hybridization, the capacitive component of the impedance between the electrodes 802, 803 is altered to a great extent.

The catcher molecules 807 are not only immobilized on the electrodes 802, 803 but also on the interspaces between the electrodes 802, 803. The particles 809 to be detected are provided with the gold labels 810 and hybridized with the catcher molecules 807. Therefore, a region within which a considerable part of the volume is filled with the metallically conductive gold labels 810 arises above the electrodes 802, 803 and in the interspaces between the electrodes 802, 803. Depending on the diameter of the gold labels 810 and depending on the density of the immobilized and hybridized molecules 807, 809, an electrically conductive connection may also arise in partial regions 811 on account of adjacent gold labels 810 making contact. However, this is not a prerequisite for the detectability of a sensor event, since the capacitive component of an impedance rather than a nonreactive resistance is detected. By way of the introduction of the material of the gold labels 810 that has good electrical conductivity, the profile of the field lines in a surrounding region of the electrodes 802, 803 is greatly influenced, that is to say that the measurement effect is large and the detection sensitivity is considerably improved.

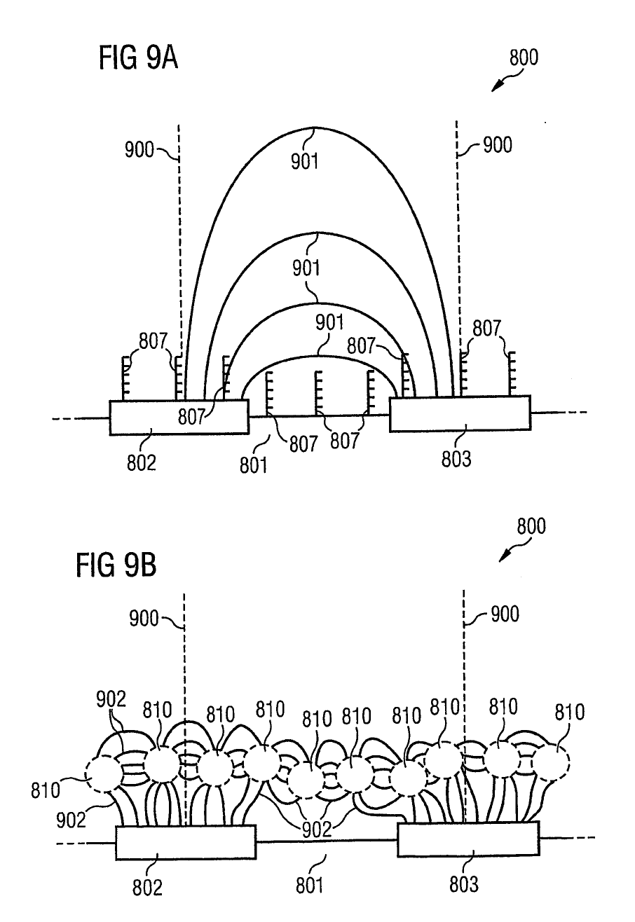

FIG. 9A schematically shows the profile of the field lines in the case of the sensor element 800 before a hybridization event. FIG. 9A shows a first profile of electric field lines 901 between lines of symmetry 900.

Furthermore, FIG. 9B schematically shows the profile of the field lines in the case of the sensor element 800 after a hybridization event that has been effected. FIG. 9B shows a scenario after an analyte 808 that has acquired the particles 809 to be detected has been brought into operative contact with the sensor element 800. After a hybridization between the catcher molecules 807 and the particles 809 to be detected (not shown in FIG. 9B), gold labels 810 coupled to the particles 809 to be detected are arranged in a surrounding region of the electrodes 802, 803, which results in a considerable distortion of the electric field lines, which is shown in the schematic second electric field line profile 902. Since the gold beads 810 are equipotential regions, the field lines 902 are orthogonal to the surfaces of the gold labels 810. A considerable densification of the field lines occurs in a surrounding region of the electrodes 802, 803, so that the capacitive component of the impedance between the electrodes 802, 803 is altered considerably on account of the sensor event.

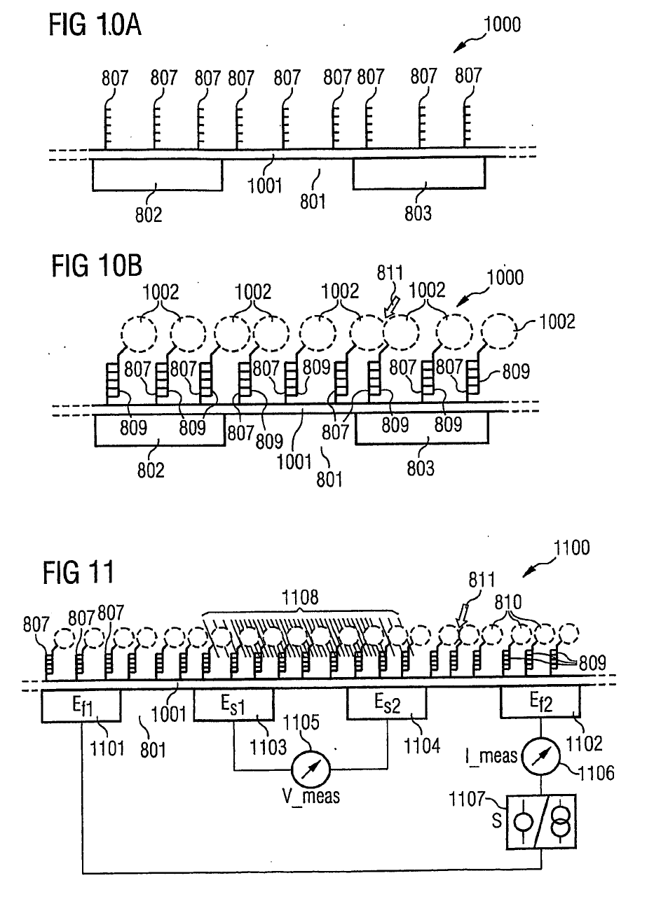

A description is given below, referring to FIG. 10A, FIG. 10B, of a biosensor element in accordance with a second example embodiment of the invention.

The sensor element 1000 shown in FIG. 10A, FIG. 10B differs from the sensor element 900 shown in FIG. 8A to FIG. 9B essentially by virtue of the fact that, instead of gold labels 810, the particles 809 to be detected have electrically insulating labels 1002, and that the electrodes 802, 803 are not arranged at the surface of the biosensor element 1000, but rather are isolated from said surface by a silicon nitride passivation layer 1001. Catcher molecules 807 are once again arranged on the passivation layer 1001 in regions above the electrodes 802, 803 and between the electrodes 802, 803. Before the biosensor element 1000 is brought into contact with an analyte possibly containing particles to be detected, the biosensor element 1000 is in the operating state of FIG. 10A.

After the biosensor element 1000 has been brought into contact with an analyte containing particles 809 to be detected, a hybridization event can take place owing to complementary base sequences of the catcher molecules 807 and of the particles 809 to be detected, as shown in FIG. 10B. In a departure from the biosensor element 800 shown in FIG. 8A to FIG. 9B, in the case of the biosensor element 1000, electrically insulating labels 102 are provided on the particles 809 to be detected.

Owing to a hybridization event, a surrounding region of the electrodes 802, 803 is thus occupied by electrically insulating labels 1002 which displace material of the electrically conductive electrolytic analyte from a surrounding region of the electrodes 802, 803. On account of the electrically insulating property of the electrically insulating labels 1002, therefore, the electrical properties in the region between the electrodes 802, 803 are significantly modified, with the result that the value of a sensor current, upon application of an electrical AC voltage signal between the electrodes 802, 803, changes significantly on account of an altered capacitive component of the impedance between the electrodes 802, 803.

A description is given below, referring to FIG. 11, of a biosensor element 1100 in accordance with a third example embodiment of the invention.

In the case of the biosensor element 1100 in FIG. 11, a first force electrode 1101 and a second force electrode 1102 are integrated in a silicon substrate 801. Furthermore, a first sense electrode 1103 and a second sense electrode 1104 are integrated in the silicon substrate 801. By way of a voltage detection unit 1105 between the first and second sense electrodes 1103, 1104, it is possible to detect a voltage between these two sense electrodes 1103, 1104. Between the force electrodes 1101, 1102, a measurement current between the force electrodes 1101, 1102 can be detected by means of a current detection unit 1106. Electrical charge carriers can be fed in by way of a charge carrier source 1107.

A silicon nitride passivation layer 1001 is provided on the electrodes 1101 to 1104 and on the regions of the silicon substrate 801 between respectively adjacent electrodes 1101 to 1104. Catcher molecules 807 are immobilized on the silicon nitride passivation layer 1001. After addition of an analyte containing particles 809 to be detected to the sensor element 1001, hybridization events are affected if the catcher molecules 807 are complementary to the particles 809 to be detected. Gold labels 810 are attached to the particles 809 to be detected. On account of the presence of the gold labels 810 having good electrical conductivity in a surrounding region of the electrodes 1101 to 1104, the electrical properties are altered and, consequently, the impedance between the electrodes is altered.

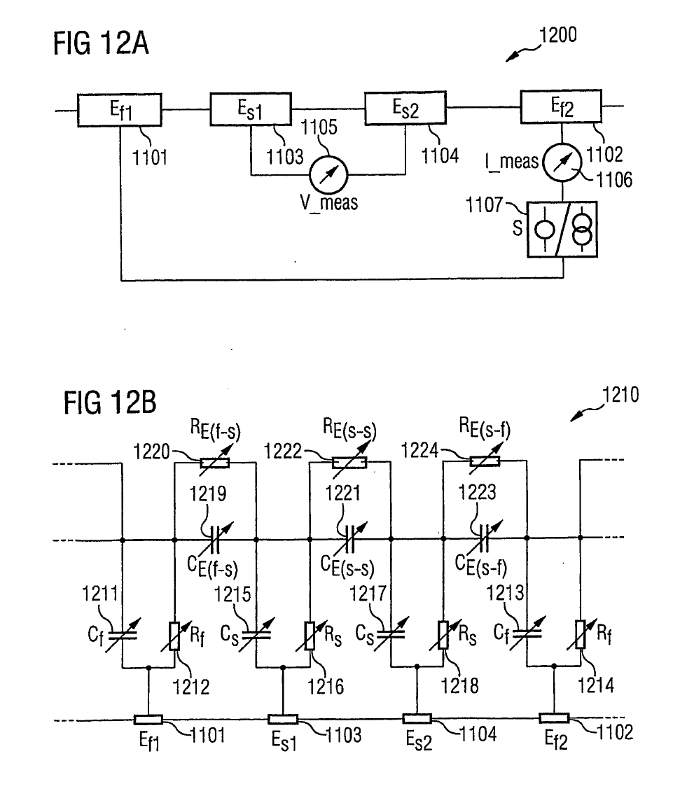

FIG. 12A shows a biosensor element 1200—modified in comparison with FIG. 11—in accordance with a fourth example embodiment of the invention without a dielectric above the electrodes 1101 to 1104.

Furthermore, FIG. 12B shows an equivalent circuit diagram 1210 with the circuitry components of the biosensor element 1200. As can be gathered from FIG. 12B, the capacitance and nonreactive resistance of the first force electrode 1101 can be modeled by way of a parallel circuit including a first force capacitance C_{f} 1211 and a first nonreactive force resistance R_{f} 1212.

Capacitances and nonreactive resistance of the second force electrode 1102 are simulated by way of a parallel circuit including a second force capacitance C_{f} 1213 and a second nonreactive force resistance R_{f} 1214. The capacitances and the nonreactive resistance of the first sense electrode 1103 are modeled by way of a parallel circuit including a first sense capacitance Cₛ 1215 and a first nonreactive sense resistance Rₛ 1216. The capacitance and nonreactive resistance of the second sense electrode 1104 are simulated by way of a parallel circuit including a second sense capacitance Cₛ 1217 and a second nonreactive sense resistance Rₛ 1218.

Furthermore, a first electrolyte capacitance C_{E(f-s)} 1219 and a first nonreactive electrolyte resistance R_{E(f-s)} 1220 connected in parallel therewith model the capacitance and nonreactive resistance of the system including first force electrode 1211, first sense electrode 1103 and the electrolyte situated in between. In an analogous manner, the second electrolyte capacitance C_{E(s-s)} 1221 and the second nonreactive electrolyte resistance R_{E(s-s)} 1222 connected in parallel therewith model the capacitance and nonreactive resistance of the system including the first sense electrode 1103, the second sense electrode 1104 and the electrolyte situated in between. The capacitance and nonreactive resistance of the system including the second sense electrode 1104 and the second force electrode 1102 and also the analyte situated in between are modeled by way of the third electrolyte capacitance C_{E(s-f)} 1223 and the third nonreactive electrolyte resistance R_{E(s-f)} 1224, which are connected in parallel with one another.

The purpose of this structure formed from force electrodes 1101, 1102 and sense electrodes 1103, 1104 is to characterize the properties of the elements C_{E(s-s) and R}_{E(s-s)}. Hybridization-dictated changes in the elements Cₛ and Rₛ, which form the entry to the measuring source, do not influence the measurement result if the inputs of the measuring source are at sufficiently high resistance. Furthermore, with the use of the four-pole principle from FIG. 11 to FIG. 12B, hybridization-dictated changes in the elements Cₛ, R_{f}, C_{E(f-s)}, R_{E(f-s)}, C_{E(s-f)} and R_{E(s-f)} are unimportant if the current which is impressed or flows into the structure and the measured voltage drop between the sense electrodes are known.

It is possible to use the sensor elements according to at least one embodiment of the invention from FIG. 11 to FIG. 12B with labels bound to particles to be detected with a four-pole method with or without a dielectric 1101 above the electrodes 1101 to 1104. As shown in FIG. 11 to FIG. 12B, the catcher molecules 807 are also immobilized in the interspaces between the electrodes 1101 to 1104. Since, in the case of successful hybridization, the majority of the field lines are forced into the volume identified by hybridization and therefore by the presence of the labels 810, in this case the four-pole method is not targeted at characterizing properties which rather are spatially associated with the volume of the electrolyte, but rather at a narrow region 1108 above the surface of the biosensor element 1100 between the sense electrodes 1103, 1104. What is advantageous about the four-pole impedance methods in comparison with a two-pole impedance method (cf. FIG. 8A to FIG. 10B) is that the electrodes themselves have no influence on the measurement result but rather essentially only the impedance between the electrodes (sensitive region 1108 in FIG. 11A).

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

The following publications are cited in this document:
[1] Paeschke, M. et al. (1996) Electroanalysis, 7, No. 1, pages 1 to 8
[2] Hintzsche, R. et al. (1997) “Microbiosensors using electrodes made in Si-technology” In: Scheller, F W et al. (eds.) “Frontiers in Biosensorics I−Fundamental Aspects”, Birkhauser Verlag Basel
[3] WO 93/22678
[4] DE 19610115 A1
[5] U.S. Pat. Ser. No. 60/007,840
[6] van Gerwen, P. et al. (1997), Transducers '97, pages 907 to 910
[7] Krause, C. et al. (1996) Langmuir, Vol. 12, No. 25, pages 6059 to 6064
[8] Mirsky, V M (1997) Biosensors&Bioelectronics, Vol. 12, No. 9-10, pages 977 to 989
[9] Thewes, R. et al. (2002) “Sensor Arrays for Fully Electronic DNA Detection on CMOS”, ISSCC Digest of Tech. Papers, pages 350f, 472f
[10] Hofmann, F. et al. (2002) “Fully Electronic DNA Detection on a CMOS Chip: Device and Process Issues”, IEDM Tech. Digest, pages 488 to 491
[11] Xue, M. et al. (2002) “A self-assembly conductive device for direct DNA identification in integrated microarray based system” IEDM Tech. Digest, pages 207 to 210
[12] 101 22 659 A1

List of reference symbols
100 Biosensor element
101 Substrate
102 First electrode
103 Second electrode
104 Partial region
200 Capture molecules
201 Analyte
202 Impedance
203 Particles to be detected
300 Surrounding regions
301 Electric field lines
302 Lines of symmetry
400 First equivalent circuit diagram
401 Second electrode-electrolyte capacitance
402 Second electrode-electrolyte resistance
403 Electrolyte capacitance
404 Electrolyte resistance
405 First electrode-electrolyte capacitance
406 First electrode-electrolyte resistance
410 Second equivalent circuit diagram
500 AC voltage source
501 Current detection unit
502 Effective electrode-electrolyte capacitance
503 Effective electrode-electrolyte resistance
504 Ground potential
800 Biosensor element
801 Silicon substrate
802 First gold electrode
803 Second gold electrode
804 Detection device
805 Buried communication line
806 External evaluation unit
807 Catcher molecules
808 Electrolytic analyte
809 Particles to be detected
810 Gold labels
811 Contact region
900 Lines of symmetry
901 First electric field line profile
902 Second electric field line profile
1000 Biosensor element
1001 Silicon nitride passivation layer
1002 Electrically insulating labels
1100 Biosensor element
1101 First force electrode
1102 Second force electrode
1103 First sense electrode
1104 Second sense electrode
1105 Voltage detection unit
1106 Current detection unit
1107 Charge carrier source
1108 Sensitive region
1200 Biosensor element
1210 Equivalent circuit diagram
1211 First force capacitance
1212 First nonreactive force resistance
1213 Second force capacitance
1214 Second nonreactive force resistance
1215 First sense capacitance
1216 First nonreactive sense resistance
1217 Second sense capacitance
1218 Second nonreactive sense resistance
1219 First electrolyte capacitance
1220 First nonreactive electrolyte resistance
1221 Second electrolyte capacitance
1222 Second nonreactive electrolyte resistance
1223 Third electrolyte capacitance
1224 Third nonreactive electrolyte resistance

1. A sensor element for detecting particles possibly contained in an analyte, comprising:
a substrate;
at least two electrodes, at least one of in and on the substrate;
catcher molecules, immobilized on a surface region of the substrate and set up in such a way that they hybridize with the particles to be detected that are possibly contained in an analyte, the particles including a label having different electrical properties than the analyte; and
a detection device, coupled to the electrodes, to detect an alteration of the capacitive component of the impedance between the electrodes on account of labels situated in a region surrounding the electrodes owing to a hybridization event.

2. The sensor element as claimed in claim 1, further comprising
an electrically insulating layer at least one of between the electrodes and the catcher molecules and on regions of the substrate between the electrodes.

3. The sensor element as claimed in claim 1,
wherein the catcher molecules are immobilized on or above the electrodes, on the one hand, and between the electrodes, on the other hand.

4. The sensor element as claimed in claim 1,
set up as a biosensor element.

5. The sensor element as claimed in claim 1,
set up as a monolithically integrated sensor element.

6. The sensor element as claimed in claim 1,
wherein the sensor element includes two electrodes, and wherein the detection device is set up for detecting an AC current signal on account of an AC voltage signal applied between two electrodes.

7. The sensor element as claimed in claim 1,
wherein the sensor element includes two pairs of electrodes, and wherein the detection device is set up for detecting a current signal at one of the pairs and for detecting a voltage signal at the other of the pairs.

8. The sensor element as claimed in claim 1,
wherein at least one of the catcher molecules are arranged at such a distance from one another and the labels have such a dimensioning that, in the case of hybridization events, the region between the electrodes is free of a continuous bridging by the labels.

9. The sensor element as claimed in claim 1, wherein
the labels are formed from an electrically insulating material.

10. The sensor element as claimed in claim 1,
wherein the labels have a relative permittivity which is greater than a relative permittivity of the analyte.

11. The sensor element as claimed in claim 1, wherein
the labels have a relative permittivity which is less than a relative permittivity of the analyte.

12. The sensor element as claimed in claim 1, wherein
the labels are formed from an electrically conductive material.

13. The sensor element as claimed in claim 12, wherein
the labels are formed from small metallic balls having dimensions in the nanometers range.

14. A sensor array comprising
a plurality of sensor elements as claimed in claim 1, are formed at least one of in and on the substrate.

15. A method for detecting particles possibly contained in an analyte, using
a sensor element including,
a substrate,
at least two electrodes, at least one of in and on the substrate,
catcher molecules, immobilized on a surface region of the substrate and set up in such a way that they hybridize with particles to be detected that are possibly contained in an analyte, the particles have including a label having different electrical properties than the analyte, and
a detection device, coupled to the electrodes, to detect an alteration of the capacitive component of the impedance between the electrodes on account of labels situated in a region surrounding the electrodes owing to a hybridization event,
the method comprising:
bringing the analyte into operative contact with the catcher molecules immobilized on the surface region of the substrate in such a way that the catcher molecules hybridize with particles to be detected that are possibly contained in the analyte, the particles including a label having different electrical properties than the analyte; and
using the detection device to detect an alteration of the capacitive component of the impedance between the electrodes on account of labels situated in a region surrounding the electrodes owing to a hybridization event.

16. The sensor element as claimed in claim 1, wherein the catcher molecules are immobilized on or above the electrodes, on the one hand, and between the electrodes, on the other hand.

17. A sensor array comprising a plurality of sensor elements as claimed in claim 2, which are formed at least one of in and on the substrate.

18. A method for detecting, using a sensor, particles possibly contained in an analyte, the method comprising:
bringing the analyte into operative contact with catcher molecules of the sensor, immobilized on the surface region of a substrate of the sensor in such a way that the catcher molecules hybridize with particles to be detected that are possibly contained in the analyte, the particles including a label having different electrical properties than the analyte; and
detecting an alteration of the capacitive component of the impedance between electrodes of the sensor on account of labels situated in a region surrounding the electrodes owing to a hybridization event.
